(19) United States
(12) Patent Application Publication
(10) Pub. No.: US 2024/0262875 A1
(43) Pub. Date: Aug. 8, 2024

(54) PROTEIN SEQUENCE DESIGN METHODS AND USES THEREOF TO PREVENT PROTEIN AGGREGATION

(71) Applicant: Brown University, Providence, RI (US)

(72) Inventor: Nicolas Lux Fawzi, Providence, RI (US)

(21) Appl. No.: 18/436,385

(22) Filed: Feb. 8, 2024

Related U.S. Application Data
(60) Provisional application No. 63/444,148, filed on Feb. 8, 2023.

(51) Int. Cl. C07K 14/435; A61K 38/00; A61K 48/00; A61P 25/28

(52) U.S. Cl. CPC C07K 14/435; A61K 48/005; A61P 25/28; A61K 38/00

(57) ABSTRACT

Provided herein are compositions and methods useful in treating diseases such as ALS and FTD. Disclosed compositions and methods can prevent protein aggregation, particularly with regard to RNA-binding proteins.

### CROSS-REFERENCE TO RELATED APPLICATION

This application claims the benefit of U.S. Provisional Application No. 63/444,148 filed Feb. 8, 2023, which is incorporated herein by reference in its entirety.

### STATEMENT REGARDING FEDERALLY-SPONSORED RESEARCH

This invention was made with government support under grant numbers NS116176 and GM147677 awarded by the National Institutes of Health. The government has certain rights in the invention.

### FIELD

Provided herein are compositions and methods for prevention of protein aggregation.

### BACKGROUND

β-sheet formation is thought to be related to aggregation of prion-like domains. Proline is an amino acid that can prevent β-sheet formation and aggregation.

RNA binding proteins are highly expressed in cells including in neurons and can be prone to β-sheet formation. However, the proteins are essential and cannot be completely “knocked out” or toxicity can result. The level of the protein is tightly maintained by RNA splicing—this is called autoregulation. Therefore, it is difficult to prevent cells from expressing these essential but aggregation-prone proteins. However, in cell culture, it is clear that expression of a heterologous protein results in decreased expression of the endogenous protein.

Gene therapy, mRNA delivery, and CRISPR are all methods that are currently in use or being developed to make cells, including in the nervous system, express designed proteins in humans.

Fused in Sarcoma (FUS), Ewing's sarcoma breakpoint region 1, TAF15, hnRNPA2, TDP-43 are related and homologous (evolutionarily related, similar properties and domain structure) proteins that aggregate in amyotrophic lateral sclerosis (ALS) and frontotemporal dementia (FTD).

The mechanism by which these proteins aggregate and the etiology of the disease is still not clear. However, these proteins contain domains or subregions that are known to be prone to self assembly and aggregation (also known as prion-like domains), as evidence by their inclusion formation in certain forms of ALS and FTD.

There is no “magic bullet” to treat neurodegenerative conditions, but the current data suggest that aggregation-prone regions contribute to toxicity. Because these domains are predicted to be unstructured, design of a small molecule to bind the region and block protein aggregation is unlikely. Therefore, a different method to reduce or eliminate aggregation by the domain is needed.

### SUMMARY

Disclosed embodiments comprise methods of reducing or preventing aggregation of proteins, for example RNA-binding proteins such as those associated with neurodegenerative conditions such as ALS or FTD.

In embodiments, protein sequences of the, for example, full-length RNA binding proteins, are made aggregation-resistant or aggregation-proof by altering amino acids, such as by adding, for example, prolines, to aggregation-prone regions of these domains, or replacing amino acids in aggregation-prone regions with, for example, prolines.

In embodiments, suitable target regions are identified, for example via aggregation-prediction tools such as zipperDB. In further embodiments, protein sequence positions that comprise specific functions (including for example the sites of post-translational modifications) can be avoided.

These modified protein versions will then be, for example, expressed via gene therapy or other means in humans, replacing the aggregation-prone natural versions with aggregation-proof or aggregation-resistant versions, and hence preventing the onset or worsening of neurodegenerative conditions such as ALS or FTD.

Thus, embodiments comprise compositions for and methods of treatment of neurodegenerative conditions such as ALS or FTD.

Further embodiments comprise models for testing scientific hypotheses regarding the role of aggregation in cellular toxicity and normal function of these proteins.

### BRIEF DESCRIPTION OF THE DRAWINGS

FIG. 1: (FIG. 1A) FUS contains a glutamine, glycine, serine and tyrosine (SYGQ) rich low complexity (LC) domain with 12 sites phosphorylated by DNA-dependent protein kinase (DNA-PK) followed by a G-rich and RGG region, an RNA recognition motif (RRM), two RGGs flanking RNA-binding RANBP2-type zinc finger domain, and a PY nuclear localization signal. FUS mutations associated with ALS (red) highlighted. R-methylation is known at >20 sites in RGG domains (pink). Example cancer fusion of FUS with DDIT3 DNA binding domain shown. (FIG. 1B) FUS LC (yellow star) has an unusual sequence composition bearing more resemblance to the FG-rich nuclear pore domains (green) than folded proteins (PDB, blue) or characterized disordered regions (DisProt, red).

FIG. 2: Molecular determinants of FUS assembly, aggregation, and interaction with binding partners.

FIG. 3: (FIG. 3A) FUS RGG3 is disordered after phase separation with FUS LC. Spectra recorded at 298K at ¹H frequency of 850 MHz. (FIG. 3B) Representative filtered/edited NOE planes showing (¹²C) ¹H positions of FUS RGG3 (G Hα, P Hγ, M Hβ, R Hγ) NOEs with FUS LC ¹³C/¹H tyrosine positions. (FIG. 3C) Many residues in FUS LC and RGG3 show NOE contacts. (FIG. 3D) Motions of RGG3 in LC/RGG3 condensate are slowed (as seen by altered relaxation rate constant values) but still consistent with significant nanosecond timescale motion (hetNOE and R1 values). Gray indicate RGG motifs. (FIG. 3E) Distribution of fluoro-tyrosine incorporation demonstrating tyrosine replacement strategy for Aim 1. (FIG. 3F) Simulated FUS RGG and LC fragment contact residue pair frequency (gray, residue types with n<3).

FIG. 4: Creation of a fully atomistic simulation of phase separated FUS domains (top left) and analysis of contact modes (bottom left) and predicted NMR observables (right) including NMR spin relaxation (top), chemical shifts which report on structure (middle), and transient contacts by paramagnetic relaxation enhancements.

FIG. 5:( FIG. 5A) Long-term liquidity of phosphoFUS droplets. (FIG. 5B) Top: ¹⁵N labeled FUS full-length protein with 12 serine to glutamate substitutions (FUS 12E) can form macroscopic liquid/flowing condensed phases after the addition of TEV protease to cleave into MBP and FUS 12E (only a small fraction of the total supernatant was transferred to this tube). Bottom: Gel shows the condensed phase is concentrated and mostly excludes MBP. CG simulations of (FIG. 5C) FUS phases without and with (FIG. 5D) polyA (test) RNA (red).

FIG. 6: Significant impact of residue substitutions on phase separation of FUS LC including (FIG. 6A) QtoA and (FIG. 6B) StoG mutations and substitution of fluoroTyr at tyrosine positions in (FIG. 6C) FUS LC (residues 1-163) and (FIG. 6D) FUS LC-RGG1 (1-284). FUS LC-RGG1 mimics much of full-length FUS LLPS behavior—high phase separation (low saturation concentration) and higher LLPS as low salt.

FIG. 7: (FIG. 7A) FUS nuclear organization and cytoplasmic stress granule formation under stress. (FIG. 7B) GFP-FUS localizes to a linear laser-damaged nuclear area in cells.

FIG. 8: (FIG. 8A) Endogenous FUS egress upon osmotic stress (0.4 M sorbitol) immunofluorescence after fixation. (FIG. 8B) FUS return to nucleus after wash out with fresh media.

FIG. 9: FUS LC wild-type undergoes LLPS at physiological salt while G156E forms irregular aggregates and S96Δ shows incomplete fusion and aggregation. 200 μM protein in 150 mM NaCl, 20 mM Tris pH 7.4. Scale bar 100 μm.

FIG. 10: FUS RGG3 shows (FIG. 10A) chemical shift changes and (FIG. 10B) enhanced LLPS upon addition of torula yeast extract RNA. (FIG. 10C) Arginine side-chain region spectrum showing new resonances associated with asymmetric dimethylation. Mass spectrum shows near complete 18 methyls added (9 RGG positions in FUS RGG3-NLS).

FIG. 11: (FIG. 11A) RNA pol II CTD localizes to FUS full-length, LC, and ΔLC droplets. (FIG. 11B) RNA pol II CTD retains disorder when in a 3-component LC+RGG+CTD phase (pink) similar to when it is alone in dispersed solution (orange, ¹H ¹⁵N HSQC spectra conducted in pH 6 20 mM MES). (FIG. 11C) Many residue types in FUS LC and RGG3 show NOE contacts with RNA pol II CTD in ¹³C-filtered/edited NOE experiments. (FIG. 11D) Quantification of NOEs observed from FUS RGG and FUS LC to RNA pol II CTD. (FIG. 11E) Summed contacts from 2 chain simulation ensembles show many LC residue types (Y, S, G, Q) and RGG residue types (R, G, Y, D) contribute contacts with all residue types in RNA pol II CTD.

FIG. 12: Observing FUS recruitment of binding partners into nuclear puncta formed at LacO arrays (and additional puncta). These data are for a fungal protein (Efg1) recruitment to FUS puncta.

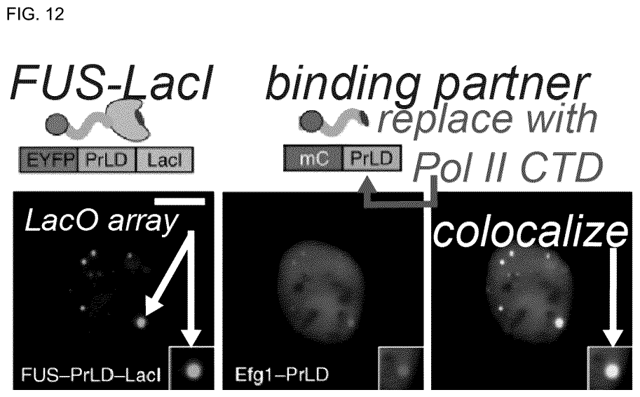

### DETAILED DESCRIPTION

Disclosed embodiments comprise methods of reducing or preventing aggregation of proteins, for example RNA-binding proteins such as those associated with neurodegenerative conditions such as ALS or FTD.

In embodiments, protein sequences of the full-length RNA binding proteins are made aggregation-resistant or aggregation-proof by altering amino acids, such as by adding prolines to aggregation-prone regions of these domains, or replacing amino acids in aggregation-prone regions with prolines.

### Definitions

“Administration,” or “to administer” means the step of giving (i.e. administering) a device, material or agent to a subject. The compositions disclosed herein can be administered via a number of appropriate routes.

“Patient” means a human or non-human subject receiving medical or veterinary care.

“Pharmaceutically acceptable carrier, diluent, or excipient” is a medium generally accepted in the art for the delivery of biologically active agents to mammals, e.g., humans. The compositions of the present disclosure can be formulated as pharmaceutical compositions or formulations using a pharmaceutically acceptable carrier, diluent, or excipient and administered by a variety of routes. Such pharmaceutical compositions and processes for preparing them are well known in the art. See, e.g., REMINGTON: THE SCIENCE AND PRACTICE OF PHARMACY (A. Gennaro, et al., eds., 19^{th} ed., Mack Publishing Co., 1995).

“Pharmaceutical composition” means a formulation including an active ingredient. The word “formulation” means that there is at least one additional ingredient (such as, for example and not limited to, an albumin [such as a human serum albumin or a recombinant human albumin] and/or sodium chloride) in the pharmaceutical composition in addition to an active ingredient. A pharmaceutical composition is therefore a formulation which is suitable for diagnostic, therapeutic or cosmetic administration to a subject, such as a human patient. The pharmaceutical composition can be: in a lyophilized or vacuum dried condition, a solution formed after reconstitution of the lyophilized or vacuum dried pharmaceutical composition with saline or water, for example, or; as a solution that does not require reconstitution. As stated, a pharmaceutical composition can be liquid, semi-solid, or solid. A pharmaceutical composition can be animal-protein free.

“Therapeutically effective amount” means the level, amount or concentration of an agent, material, or composition needed to achieve a treatment goal.

“Treat,” “treating,” or “treatment” means an alleviation or a reduction (which includes some reduction, a significant reduction, a near total reduction, and a total reduction), resolution or prevention (temporarily or permanently) of a symptom, disease, disorder or condition, so as to achieve a desired therapeutic or cosmetic result, such as by healing of injured or damaged tissue, or by altering, changing, enhancing, improving, ameliorating and/or beautifying an existing or perceived disease, disorder or condition.

Recent work has linked a family of RNA-binding proteins containing aggregation-prone putatively disordered domains to the formation of membrane-less organelles, oncogenic fusion proteins, and neurodegenerative disease-associated aggregates.

Fused in Sarcoma (FUS) has emerged as the archetype of a large family of human multifunctional RNA processing proteins that contain polar-residue-rich low complexity (LC) domains, RGG domains (regions enriched in the RGG sequence motif), and folded RNA binding domains.

Each domain plays a role in the formation of dynamic assemblies and puncta in cells associated with function in transcription, splicing, cellular stress, and DNA damage response. FUS domains also play important roles in disease; first, chromosomal translocations of FUS (and two paralogs, EWS and TAF15) create fusions of the FUS LC (and sometimes longer pieces) with one of several transcription factors (DNA-binding domains) resulting in potent transcriptional activators causing forms of leukemia and sarcoma.

Second, FUS is one of dozens of RNA-binding proteins forming intracellular neuronal inclusions causally linked to both ALS and FTD. Relationships between FUS assembly and function as well as disease have been established, however, the mechanisms linking phase separation and physiological function and the disease “switch” for FUS is only beginning to be understood with structural detail. This knowledge gap results from:
a. incomplete residue/atomic-level view of how the domains of FUS each contribute to functional liquid-liquid phase separation (LLPS) and disease aggregation; and
b. lack of studies tying the structural details to in cell behavior, function, and dysfunction.

These states are invisible to traditional structural biology due to the difficulty in observing phase separated and aggregated states, yet mechanistic details in vitro are needed to guide and link to work in cells and ultimately in vivo.

Disclosed embodiments comprise methods of reducing or preventing aggregation of proteins, for example RNA-binding proteins such as those associated with development or progression of, for example, neurodegenerative conditions such as ALS and FTD.

In embodiments, suitable regions are identified, for example via aggregation-prediction tools including zipperDB.

In embodiments, protein sequences of the full-length RNA binding proteins are made aggregation-resistant or aggregation-proof by altering amino acids, for example by adding prolines to aggregation-prone regions of these domains, or replacing amino acids in aggregation-prone regions with prolines.

For example, in embodiments, the proline content of a given amino acid sequence is as compared to wild type increased by 5%, 10%, 15%, 20%, 25%, 30%, 35%, 40%, 45%, 50%, 55%, 60%, 65%, 70%, 75%, or the like.

In embodiments, the proline content of a given amino acid sequence is as compared to wild type increased by at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, or the like.

In embodiments, the proline content of a given amino acid sequence is as compared to wild type increased by not more than 5%, not more than 10%, not more than 15%, not more than 20%, not more than 25%, not more than 30%, not more than 35%, not more than 40%, not more than 45%, not more than 50%, not more than 55%, not more than 60%, not more than 65%, not more than 70%, not more than 75%, or the like.

In embodiments, these modified versions will then be, for example, expressed via gene therapy or other means in humans, replacing the aggregation-prone natural versions with aggregation-proof or aggregation-resistant versions, and hence prevent the worsening or onset of ALS or FTD.

In further embodiments, protein sequence positions that enable specific functions including the sites of post-translational modifications can be avoided.

Thus, embodiments comprise compositions and methods of treatment of ALS or FTD.

### EXAMPLES

### Example 1

The objective was to bring the entirety of FUS into focus both in atomic detail and complemented by assays in cells, evaluating both functional and pathological FUS assemblies and regulatory mechanisms. FUS served as a model for an entire class of RNA-binding proteins associated with disease. Importantly, molecular details of even the basic interactions between the domains of this highly-studied protein are still being debated.

The studies tested the central hypothesis that biological function and pathological dysfunction of FUS associated with phase separation and aggregation can be controlled by sequence including post-translational modifications and mutations.

This hypothesis was based on sequence proximity of imperfect repeats that contribute to LLPS and aggregation ALS-associated missense mutations, and physiologically functional modifications, but details remained unclear.

### Dissecting the Molecular Interactions of Disordered RNA Binding Proteins in FUS Assembly.

Because FUS can form fibrillar structures and because some of the amino acid types/patterns that contribute to LLPS have been uncovered, competing views that either β-sheet segments or a minimal set of disordered molecular interactions (e.g. cation-π) lead to LLPS remain debated. Our preliminary data showed that a full picture of disordered protein LLPS is still developing. The current work probes residues and contacts contributing to multi-modal disordered LC and RGG LLPS. This defines interactions driving FUS assembly and also directly tests the correspondence between in vitro LLPS and in cell FUS assembly and function.

### Determine the Effects of Disease-Linked Mutations on FUS LLPS in Vitro and in Cells.

Using solution NMR, computer simulation, and cell models of FUS function and dysfunction, we evaluated the mechanism by which FUS mutations outside the FUS nuclear localization signal induce aberrant assembly and hence disease.

FUS Domains Cooperate in Contacts with Binding Partners.

Our data demonstrated that FUS LC and RGGs form contacts with RNA Pol II CTD while FUS RGGs mediate contacts with RNA. We probed the multivalent contacts formed between FUS and the CTD that are associated with transcriptional function and cancer and the interaction and LLPS of FUS with RNA that is associated with RNA splicing and RNA granule formation. A structural view of FUS contacts with binding partners in physiological context provides insights about how FUS executes its normal RNA processing functions and its role in transcriptional activation in cancer.

FUS forms functional and pathological interactions which can serve as important models for the large family of RNA-binding proteins with similar domains. Because these sequences mediate higher-order assemblies in physiological phase separation and disease aggregation, a molecular understanding of these structures is necessary to find ways to modulate FUS self-association in disease.

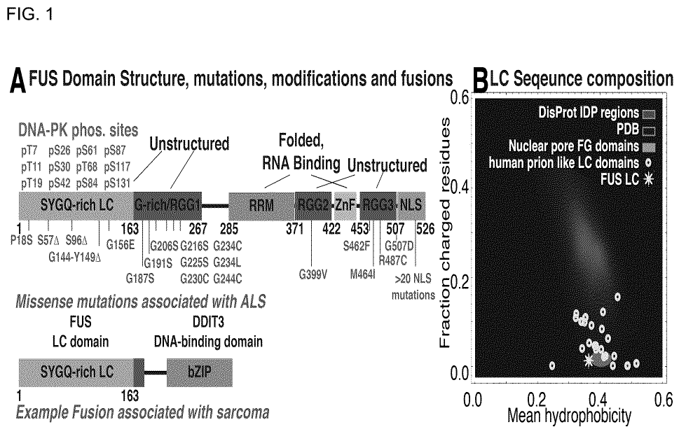

A broad array of complex, poorly understood functions in eukaryotes is associated with higher order assembly of RNA-binding proteins mediated by structurally disordered domains, many of which have a repetitive, low complexity (LC) sequence and are prone to self-association. For example, LC domains of RNA-binding proteins code for tunable self-association, facilitating mating in yeast by the dynamic super-assemblies of the protein Whi3, regulating learning-associated synaptic plasticity by the assembly of cytoplasmic polyadenylation element-binding protein in Aplysia sea slugs, and coordinating RNA processing by the diverse family of heterogeneous nuclear ribonucleoproteins (hnRNPs) in animals. In the human hnRNP Fused in Sarcoma (FUS) (FIG. 1A), the low complexity (LC) N-terminal domain and the RGG domains together mediate contacts critical for transcriptional regulation, pre-mRNA splicing, and mRNA transport and stability. Composed primarily of serine, tyrosine, glycine, and glutamine (SYGQ-rich) and nearly devoid of aliphatic and charged residues, the LC domain of FUS and 28 other human RNA-binding proteins resemble the sequence composition and aggregation propensity of yeast prion proteins.

Hence, these LC domains are also referred to as “prion-like” and are distinct in sequence composition from most disordered proteins (FIG. 1B). FUS, like many of these hnRNPs (and as many as 1000 human proteins) also contains arginine-glycine-glycine sequence repeat motifs (RGG domains/“RGGs”) that aid in RNA-binding as well as protein-protein interaction and are targeted for arginine methylation, which is reduced in neurodegenerative disease. Recently, hnRNP function and dysfunction have been linked to liquid-liquid phase separation into membrane-free punctate structures known as granules, membrane-less organelles, or biomolecular condensates.

FUS has served as a primary model for understanding the role of protein disordered domains and interactions that mediate phase separation and aggregation in RNA-binding protein function and dysfunction. Critically, the physiological importance of the LC and RGG domains of FUS has been rigorously established. FUS LC and RGGs are essential for functional FUS self assembly into transcriptional chromatin-associated puncta in the nucleus and RNA granules in the cytoplasm. FUS LC and RGGs both cooperate in the aggregation of FUS into protein inclusions in ALS and FTD. FUS or two other human paralogs, RNA-binding protein EWS and TATA-binding protein-associated factor 2N (product of TAF15 gene), are also translocated in cancers.

These chromosomal translocations fuse the LC domain and sometimes additional domains of FUS, EWS, or TAF15 to one of several DNA-binding domains. These FUS-family disordered domains then act as strong transcriptional activators leading to nine forms of related sarcomas and leukemias. Furthermore, the information about how these normal and disease processes are regulated in cells is emerging. For example, despite being small chemical changes, serine/threonine phosphorylation and arginine methylation of FUS disordered domains critically impact FUS biophysical, biological, and pathological processes.

Therefore, the biological and pathological importance of FUS and a large family of proteins, and specifically the disordered domains interactions, has been established, but the mechanistic details have been sparse.

Here, we provide a full and directly detailed picture of the molecular interactions that mediate contacts important for phase separation and physiological assembly, a molecular understanding of how LC and RGG mutations contribute to disease, and a picture of FUS domain interactions associated with RNA processing and transcription. This contribution is significant because atomic resolution insight into FUS self-association resulting from this project is a critical step in:
understanding the molecular function of these domains that are not amenable to classic structural biology or biochemistry; and
designing new therapies for both rapidly progressing, fatal neurodegenerative disease (ALS and FTD) and the aggressive cancers associated with FUS and its homologs for which effective treatments are currently lacking.

Determining how FUS self-interacts physiologically and pathologically, with atomic detail and in coordination with work in cells, provides broadly useful knowledge for targeting therapies aiming to alter condensate formation in cancer and reducing aggregation in neurodegenerative disease. Furthermore, understanding the biophysical changes caused by disease-causing mutations provides information not only on the hereditary forms of the disease but also on the pathways common with the clinically-indistinguishable sporadic forms. Because a large family of proteins share similar domain composition and also play roles in disease, understanding the structural details of FUS self-interaction and partner-binding will be a broadly relevant model for mechanism and dysfunction.

Before FUS phase separation was first explored, FUS LC had been shown to form fibrillar gels stabilized by β-sheet structures-hence, it was hypothesized that FUS LLPS is primarily β-sheet driven. Indeed, FUS and many related proteins do aggregate in disease and contain many regions identified as aggregation-prone low complexity aromatic-rich kinked segments (LARKS) that can stabilize fibril forms. Despite our prior and recent data (direct observation of FUS liquids) showing that FUS LC can remain predominantly disordered in liquid phases, this β-sheet hypothesis has been extended to include the physiological assembly of other important proteins in RNA processing and ALS, such as hnRNPA2 and TDP-43.

At the same time, another popular model for explaining phase separation of these domains is the “stickers and spacers” model. A highly simplified version of this model has become popular wherein only certain residue types provide interactions, thereby driving phase separation by interacting with other “stickers” favorably (i.e. Y-Y and Y-R contacts) using certain contact modes (e.g. cation-π and π-π interactions). The remaining enriched residue types (Q, G, S, P for FUS) are thought to only/primarily modulate aggregation-propensity or other functional features and other interaction modes (e.g. hydrophobic contacts, hydrogen bonds) do not make primary contributions.

Our work reveals deficiencies in the “β-sheet driven” model while providing important additions to the “stickers and spacers” model for FUS phase separation. Though FUS can form β-sheets, our work shows how LARKS do not explain FUS phase separation. At the same time, while Y and R are certainly major contributors, these residues do not only interact with themselves. In addition, they do not interact via only cation-π/π-π contacts—other residue pairs and contact modes modulate phase separation. Hence, an important unifying theme throughout this work is how all the residues contribute to phase separation and interactions in FUS. In summary, this contribution is significant because the molecular details revealed here are essential to predicting FUS phase separation, FUS functional contacts, as well as how mutations alter

FUS in disease. This is all the more important because of the shared sequence features in many RNA-binding protein disordered domains—hence these data will provide important molecular details governing disordered protein phase separation more broadly—and this information is necessary for future design of phase separating materials and therapeutic modulation of phase separation and aggregation.

The application of new methods to visualize FUS assembly is critical to reveal how association of FUS with itself and other proteins occurs. The outcomes of this research will guide future therapeutic strategies aimed at disrupting pathogenic assembly of FUS and related proteins in cancer and neurodegenerative disease such as ALS and dementia. Our previous studies have focused on the low complexity domain of FUS and its role in phase separation and aggregation. Here, we make an important expansion to examine the contacts formed by the entire protein as well as probe the partner molecular interactions formed by FUS. A large number of studies have demonstrated the importance and contribution of the various domains of FUS in phase separation, function, and aggregation in vitro and in cells. However, these approaches are not able to provide a mechanistic understanding of FUS assembly, as they are limited to observing phenotypic and biochemical changes or arrangements limited by the current techniques (e.g., in light microscopy). For example, co-recruitment of FUS and RNA polymerase II CTD has been observed by fluorescence microscopy and associated with transcription initiation, but structural details of this dynamic and disordered interaction cannot be evaluated by x-ray crystallography or cryo-EM, which require some molecular order. Importantly, we have demonstrated that liquid-liquid phase separated forms of FUS and related proteins can be visualized by solution NMR.

Here, we will directly observe the role of all domains of FUS in FUS assembly and aggregation by combining the strengths of the primary approaches (solution NMR and molecular simulation) with Raman spectroscopy and solid-state NMR. As of yet, there are no reports of NMR spectroscopic detail of intact proteins (with folded domains) in a liquid phase model, nor any reports of how RNA interacts with disordered and folded regions in these phases. The research is innovative because we have for the first time observed new contacts in FUS and seen folded domains in phase separated systems. We have overcome resolution limitations by applying solution NMR techniques to faithful models of the intact FUS protein in complex with known binding partners. Further, we have examined the effects of ALS-linked mutations located outside the FUS NLS, mutations which are largely understudied in the ALS field, to gain mechanistic insight into how these amino acid substitutions alter FUS LLPS and how altered LLPS effects FUS function in cells. Pairing state-of-the-art experimental studies with computational structural biology techniques and cell models available in the laboratories of the multidisciplinary team, we have been able to test atomic-level structural hypotheses in cellular and functional assays, linking the observed behavior to protein sequence/interaction changes to explain the observed effects.

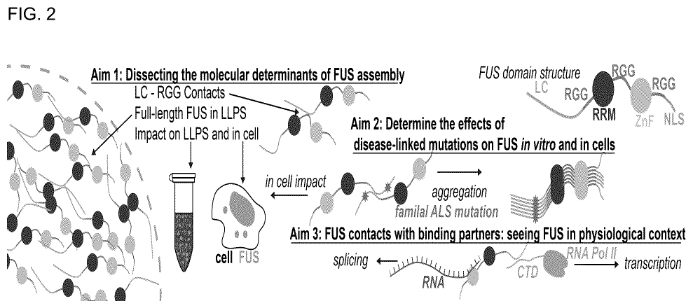

These studies (summarized in FIG. 2) provide a mechanistic view of FUS structure and function that no other techniques offer. Because disease-associated mutations demonstrate that even a single amino-acid substitution can cause ALS and FTD, seeing FUS interactions with residue-by-residue resolution will provide necessary and critical insights into function and dysfunction of FUS and provide novel avenues for therapies.

### Dissecting the Molecular Determinants of FUS Assembly:

Understanding how dynamic nuclear and cytoplasmic puncta enriched in FUS and related RNA binding proteins direct RNA transcription and processing requires a molecular level picture of the interactions and phase separation of these multidomain proteins. The objective was to create a mechanistic picture of FUS-FUS interactions that resulted in liquid-liquid phase separation in vitro and functional FUS-FUS assembly in cells. We tested our working hypotheses that FUS phase separation is stabilized by disordered LC and RGG repeats forming a network of multimodal contacts spanning all the enriched residue types in LC and RGG, and that in vitro LLPS predicts in-cell FUS assembly. The rationale for these studies was that a detailed description of structure and contacts formed by FUS will provide knowledge on how these unusual, repetitive sequence regions function across a large family of RNA-binding proteins. Furthermore, before tackling the effect of disease mutation and the complex cellular environment, it is important to build towards residue-level characterization of all the domains of FUS in in vitro models that are already established as faithful mimics for many aspects of in-cell FUS assembly.

We provided new insight into how FUS sequence controls in-cell assembly using in-cell visualization of FUS variant puncta. The fundamental approach will be to:
directly visualize the contacts important for interaction and phase separation of the LC and RGG domains;
provide the first visualization of the interplay of the disordered and folded domains of the full-length protein upon phase separation; and
examine the impact of the observed contacts on FUS self-assembly in vitro and in cells.

Together these pieces will make a complete and molecularly detailed picture of the contacts formed by the multiple parts of FUS that mediate its physiological assembly.

### Justification and Feasibility:

Despite the many roles of FUS in RNA processing, atomic-level structural details essential to understand its function are only beginning to emerge. Previously, we demonstrated that FUS LC is predominantly disordered both as a monomer and within in vitro phase separated droplets. Similarly, FUS RGGs are predicted to be disordered based on their glycine-rich sequence. Based on recent biophysical, biochemical, and cellular studies, conserved RGG and/or LC regions are required both for cytoplasmic (stress granule recruitment) and nuclear (DNA damage site puncta formation) FUS function in these stress responses. Dynamic association is therefore a fundamental role of these domains, precisely controlled in normal function but dysregulated in FUS associated cancers and in neurodegenerative diseases. Importantly, a “molecular grammar” specifying the interactions made by the LC and RGG domains of FUS has been proposed, based on mutagenesis studies altering phase separation. Yet, these mutagenic approaches have focused only on a few residue types and cannot distinguish which contacts these key residues actually form.

Direct residue-by-residue observation of FUS disordered domain interactions has been achieved only for FUS LC (by our laboratory) and is not available for the other domains. FUS RGG domains are essential for in vivo formation of dynamic functional complexes and FUS incorporation into membrane-less organelles such as stress-granules and transcriptionally activating nuclear assemblies. High correspondence between in vitro and in cell assembly of FUS cytoplasmic and nuclear phase separated structures suggests that studying FUS assembly in vitro can yield important insights into the contacts and structured formed in cells. However, to understand how FUS forms self-interactions, it is crucial to determine residue-by-residue details of these dynamic phase separated assemblies, for which our combination of approaches is ideally suited.

### Research Design and Preliminary Studies:

### Visualizing FUS RGG and LC Molecular Contacts in Phase Separation:

Direct observation of contacts between FUS LC and FUS RGG is essential for understanding the molecular architectures undergirding FUS phase separation -mutagenesis can be used to determine which residues are forming important interactions but cannot directly determine structural features or interaction pairs. Our combined experimental and computational approach visualizes and validates the structured and contacts formed by FUS LC and FUS RGGs as important models for the large protein family containing these domains.

### NMR of FUS RGG Domains:

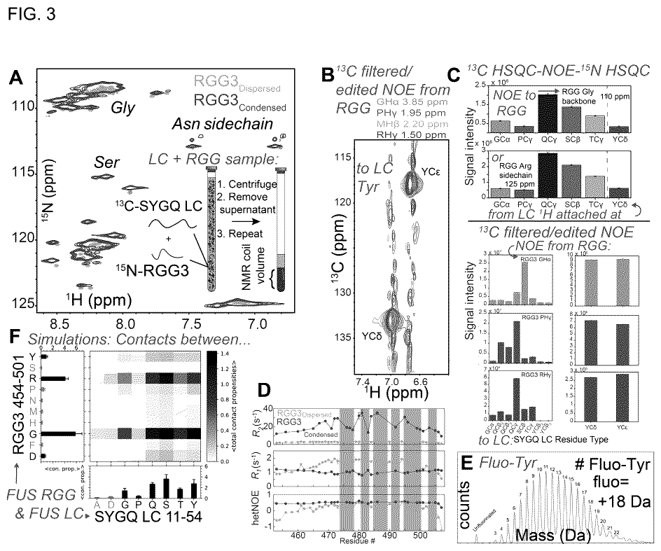

We have directly visualized the structural features and position-specific as well as residue-residue contact details of phases containing both FUS LC and FUS RGGs. To this end, using our established combination of standard 3D triple resonance NMR experiments optimized for repetitive disordered domains (HNCO, HNCAO, CBCACONH, HNCACB, high ¹³C resolution HNCA, HNN), we obtained complete resonance assignment of non-proline residue positions in FUS RGG1, RGG2, and RGG3 in the dispersed phase. These data indicate that RGGs are structurally disordered (FIG. 3A).

### Structure and Local Motions:

By generating macroscopic co-condensed phases of FUS RGG3 and FUS LC, our preliminary data provided support for the hypothesis that RGG3 (chosen for its better resolved HSQC spectrum) and FUS LC both remain predominantly disordered in the mixed condensed liquid phase (FIG. 3A), showing retention of fast motions (FIG. 3D). To test for the presence of structured conformations especially in the RGG domains that may be obscured due to slow motions or chemical exchange, we used our published combination of condensed-phase relaxation dispersion and dark-state exchange transfer NMR (to test for exchange with structured conformations and interactions with ordered forms, respectively) and Raman spectroscopy with Sapun Parekh (to provide a structural fingerprint that is not impacted by molecular motion). We used a magic angle spinning (MAS) ssNMR approach on liquid phases of FUS LC now applied to mixed phases with either LC or RGG ¹⁵N/¹³C labeled to probe for rigid structure/disorder using cross polarization (CP) and INEPT experiments, respectively.

### Contact Types:

To probe transient molecular contacts, our preliminary data include similar 3D filtered/edited NOESY experiments as our work on FUS LC to test the hypothesis that residue types beyond only tyrosine and arginine mediate LC/RGG contact. Our data show evidence for molecular contacts between LC and RGG domains in the phase. These NOEs report on transient contacts between all the enriched residue types (i.e. LC: S/Y/G/Q/P, RGG: R/G) (FIGS. 3B,C) and not solely between R-Y pairs as might be expected from prior mutational work (but in accordance with our observed NOEs for FUS LC condensed phases and those for DDX4). Contacts are expected to be transient (<1 μs lifetime) given the motions retained (FIG. 3D), though NOEs are likely of sufficient signal due to high concentration in the phase. To rule out spin diffusion contributions to the network of NOEs, we will 1) perform NOE build-up experiments, 2) measure rotating frame Overhauser effects (ROE) which remove spin diffusion and 3) use selective spin inversion (e.g. aromatic vs aliphatic ¹H) during NOE transfer. Together these data provide a comprehensive NMR interaction view of the network of interactions between residue types within the phase.

### Contact Locations:

We complemented this residue-level picture by examining which regions of FUS LC (N-terminal, central “core”, and/or C-terminal) and FUS RGG3 (polyglycine or RGG-motif region) mediate contacts (interaction “hotspots”) using 1) paramagnetic relaxation enhancement in analogy to our previous work on FUS LC in the condensed phase and 2) position-specific backbone ¹HN NOEs using ²H/¹⁵N+¹H¹³C (in ¹H₂O) mixed samples as recently shown for a similar phase separated state. Together these data will show how and via what residues and positions FUS forms disordered domain contacts that stabilize phase separation.

### Alternative Strategies:

If NOE build-up curves are ambiguous and ROEs unattainable due to fast transverse relaxation, we would instead rule out tyrosine-driven spin diffusion by performing NOE experiments on samples where tyrosine residues are highly and specifically deuterated (to remove its H positions from the NMR relaxation matrix) using glyphosate inhibition of aromatic residue synthesis already applied for fluorinating similar domains that we have used to incorporate fluorotyrosine into FUS LC (FIG. 3E) and test if the NOEs between non-Tyr positions are still present.

### Molecular Simulation:

We complemented this experimental view with a novel picture of FUS LC and FUS RGG phases at atomic detail (with explicit solvent molecules) using advanced molecular simulations and all-atom protein models we refined based on detailed comparison to NMR data from 4 different LCs (FUS LC, hnRNPA2 LC, TDP-43 CTD, RNA Pol II CTD) to better represent the residue-level helical propensity in domains like FUS LC that are enriched in these polar residues. Our preliminary data on simulations of one LC fragment and one RGG fragment show contacts mediated primarily by the enriched R and G residues as well as the Y and D residues of RGG to many residue types in the LC, not just Y (FIG. 3F).

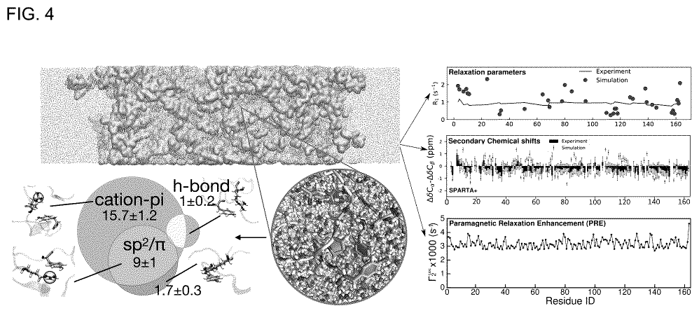

We scaled up to phase separated fully atomistic condensed phase slab simulations containing dozens of copies of each chain, as we have recently described for LC alone. Briefly, equilibrated structures from our coarse-grained simulated condensed phases of FUS LC and RGG3 will be transformed to all-atom resolution using PULCHRA, which has been tested on a set of high-resolution crystallographic structures. Our published data on FUS LC alone (FIG. 4) show that a few microseconds of condensed phase trajectories, generated within a few weeks using standard supercomputing resources (GPUs) or a few hours with the ANTON supercomputer, is sufficient to obtain high-quality comparisons to experimental observables such as NMR relaxation (top and chemical shifts for validation, and sought-after information on molecular contact, interaction modes, and sites of interaction in the condensate.

### Visualizing Full-Length FUS Within in Vitro Granules:

Structures and contacts; We and others have used NMR to directly observe the contacts between, and motions of, disordered domains of RNA-binding proteins directly in condensed phase samples. We have also used NMR to compare the LC domain and folded domains to the full-length protein. Though folded domains play a role in LLPS, their influence on the contacts formed by the disordered domains and the contacts they form in LLPS has yet to be probed.

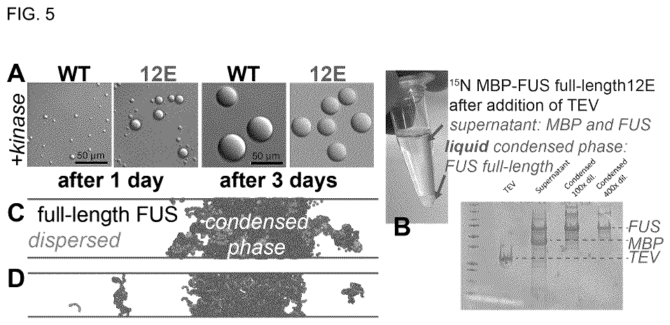

NMR; To directly probe the interactions and motions of the full-length FUS in granules previously studied extensively by microscopy in cells and in dilute solution but never observed directly, we created in vitro macroscopic condensates of full-length FUS. An issue here is the creation of samples of FUS that are sufficiently stable for multi-day NMR experiments. Importantly, we recently showed that physiological phosphorylation using in vitro DNA-PK or mutation of only 6 or 12 serines to phosphomimetic glutamic acid (FUS 6E or 12E) keeps full-length FUS liquid even under agitation, with or without addition of a total yeast RNA extract (FIG. 5A). We have also recently shown that addition of proline residues in specific places leaves FUS LC LLPS unchanged but abrogates aggregation.

We have now generated large volume samples of FUS full-length for NMR. In brief, we have expressed our established, soluble, MBP-FUS (wildtype phosphorylated, 6E, or “+proline” aggregation-resistant) and cleaved MBP after purification to generate droplets. Droplets were be fused by gravity or centrifugation. Initial samples were ¹⁵N-labeled only to optimize conditions and ¹H,¹⁵N HSQC experiments spectra were compared to our previous spectra of a soluble form of full-length FUS monomer.

Assignments for each domain are available and we have demonstrated they are sufficiently resolved for reasonable coverage even in near full-length constructs. If resonance positions shift such that transferring assignment is not clear, we will confirm assignments within the phase by triple resonance NMR or ¹⁵N HSQC-NOESY-¹⁵N HSQC backbone walk we used previously for condensed phase FUS LC43.

Once conditions were optimized, ²H/¹⁵N/¹³C FUS samples were examined with ¹H ¹⁵N TROSY-based fingerprint and NMR spin relaxation experiments to determine where any structural changes occur (based on NMR chemical shift changes) and the motions of FUS with residue-by-residue resolution. To test the hypothesis that LC, RGG, and folded domains form a network of contacts in condensed phases of full-length FUS, we used our previously published approach of paramagnetic relaxation enhancement NMR within the condensed phase. To this end we made single cysteine substitutions in cysteine-free version of FUS (excepting the ZnF cysteines that stably coordinate zinc) to introduce site specific paramagnetic tags in a small fraction of the proteins used to create the sample.

These labels allowed measurement of transient close intermolecular approach of the spin-labeled region with the regions (both ordered and disordered) that show spin relaxation. Importantly, we also compared the contacts visualized for full-length FUS in the presence and absence of RNA extract that stimulates phase separation.

Data on FUS will provide 1) a structurally detailed picture of what FUS assemblies look like, what contacts are made, how are folded and disordered domains arranged and move in full-length protein condensates and 2) serve as a template for future work in other protein systems.

### Alternative Strategies:

If samples did not retain sufficiently stable liquid forms, we would explore how in vitro methylation, addition of TMAO co-solvent, or addition of β-sheet breaking proline residues in the LC (see below) could further discourage full-length FUS transition to aggregation. Though individually small and visible by ¹H ¹⁵N NMR, the folded domains of

FUS may tumble too slowly in the condensed phase environment to be visualized by backbone ¹H ¹⁵N NMR approaches. In that case, we will observe the folded domains/contacts using ¹H ¹³C methyl labeling strategies on perdeuterated (and ¹⁵N) background to optimize samples for methyl TROSY detection as the PI has previously performed. Given that FUS LC and RGGs are depleted in methyl residues, we anticipate that spectra will be sufficiently resolved and assignable domain-by-domain in solution (dispersed phase).

### Alternative:

If N/H resolution is limiting, we would work to make segmentally labeled samples of FUS, as shown previously. Simulations: We have begun CG simulations of full-length (folded+disordered domains) FUS to identify inter-residue contacts and help interpret PRE data (FIGS. 5C,D). Together, these experimental and simulation data will be a large step forward in observing the mechanistic behavior of a full-length RNA binding protein in phase-separated assemblies and the role of RNA in organizing protein-protein contacts.

Testing impact of the contacts on LLPS in vitro as well as phase separation and function in cells

In vitro: Here we test the role of 1) specific residues/contacts and 2) LARKS/β-sheet contacts on FUS phase separation. For residue types that are shown to form contacts in the dense phase by NOEs and residue regions showing contacts by PRE, we use mutation to test the significance of these contacts and regions to phase separation.

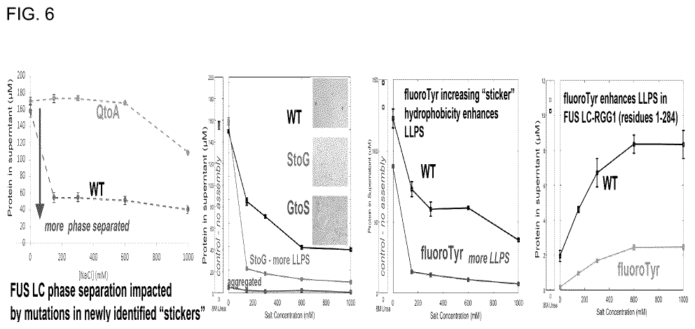

Importantly, we already identified that removal of glutamine residues disrupts in vitro phase separation of FUS44. We now show that changing all glutamine to glycine has no effect while glutamine to alanine abrogates LLPS at these conditions-hence Q, G, S, and A are not playing equal roles as “spacers” (FIG. 6). Changing all FUS LC serine to glycine dramatically enhances LLPS while changing all glycine to serine leads to aggregation without LLPS, suggesting a balance of effects. We tested the role of threonine (10 in FUS LC) by changing to serine (smaller but preserves hydroxyl) and valine (isosteric but removes hydroxyl). It is well known the Tyr to Phe decreases FUS LLPS. Our preliminary data show fluoroTyr greatly enhances LLPS (FIG. 6B), unlike fluoroPhe which decreased DDX4 LLPS. We now tested additional substitutions (e.g. chloro-and aminoTyr) and substitutions that directly alter Tyr's OH (aminoPhe, nitroPhe) to probe tyrosine's unique interactions. As for the RGG regions, based on preliminary computational work showing Y and D residues in RGG3 mediate contacts (FIG. 3F), we will test the role of conserved Y and D (e.g. Y to S, D to S) in the RGG, providing insight on aromatic and negatively charged residue contributions to the RGG domain.

Using these constructs to probe the correlation between sequence, single-protein biophysical behavior, and LLPS, we will measure the effect of these variants on LLPS saturation concentration using our established approaches. Importantly, we will perform these experiments on the appropriate combination of constructs: FUS LC, FUS LC-RGG1 (an important minimal model for full-length protein which shows less phase separation with increasing salt concentration, opposite that of FUS LC), as well as FUS full-length so that we can provide new information on how these mutations impact both intra-and inter-domain interactions.

### β-Sheets in LLPS:

To further test the hypothesis that intermolecular β-sheet formation by LARKS contributes to phase separation of FUS LC, we introduced (added) 12 prolines at sites predicted to be prone to β-sheet assembly by zipperDB (the definition of LARKS). Using our published saturation concentration and microscopy approaches, our preliminary data suggest LLPS of FUS LC is not affected by addition of LARKS-disrupting prolines while aggregation is prevented. We next tested this LARKS-disrupting version of FUS LC in full-length FUS phase separation and aggregation using our established approaches, and in cell assembly and functional assays described below.

In cell Phase Separation and Function:

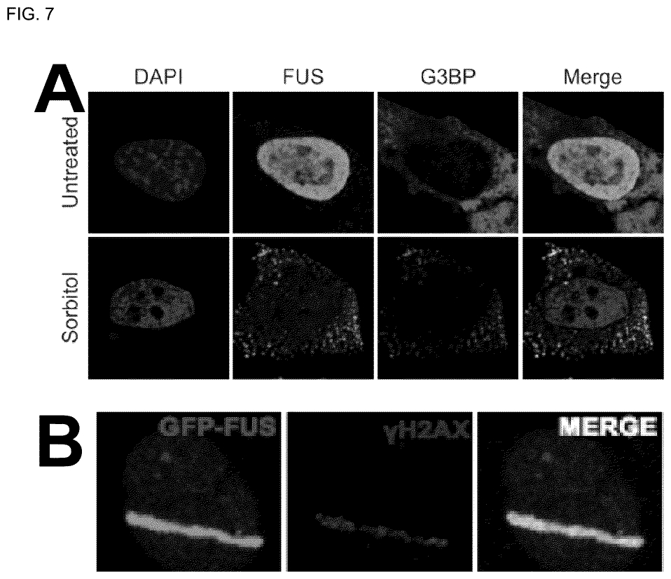

The above data provided mechanistic details in vitro, but proposed contacts and interactions must be tested to ensure they represent functional, in-cell phase separation of FUS. Therefore, we collaborated with an expert in ALS and FUS cell biology. Others have demonstrated that FUS predominantly localizes to the nucleus under homeostatic conditions, where it exhibits a widespread yet punctate pattern (FIG. 7A, top). LLPS of self-assembled FUS likely contributes to the formation of these nuclear puncta. Within minutes of cells undergoing stress, FUS rapidly redistributes and self-assembles into other condensates, including cytoplasmic stress granules in response to hyperosmolar stress (FIG. 7A, bottom) and sites of DNA damage in the nucleus (FIG. 7B).

Notably, the association of FUS with these condensates involves the FUS LC and/or RGG domains. Further, FUS is believed to play critical roles in both DNA damage and stress response in cells. Therefore, these in-cell studies were ideal for interrogating our LLPS-disrupting mutations, and will provide new insight into the residues of FUS that are essential for FUS assembly (and potentially disassembly) in cells—as well as an important test for the hypothesis that in vitro LLPS predicts in-cell assembly and functional behavior. Starting with the 8 variants that had most impact on LLPS as well as the QtoG variant and +proline variants that have surprisingly little impact on LLPS in vitro, GFP-tagged FUS constructs was transfected into HeLa cells, and FUS nuclear and cytoplasmic expression will be assessed with fluorescence microscopy. DNA damage was induced using a Nikon A1 confocal system with 405 laser, and the association of GFP-FUS will be quantified as described.

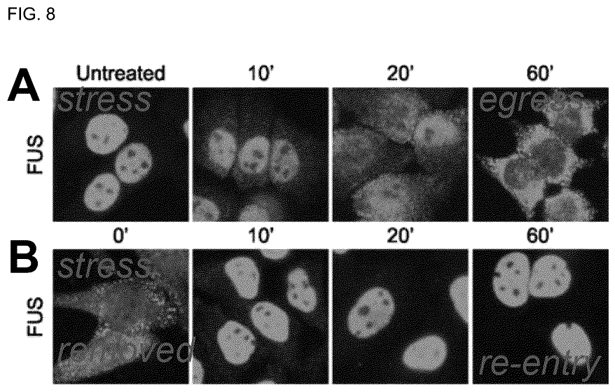

In separate experiments (FIG. 8), we examined the egress to the cytoplasm and formation of stress granule condensates, as well as their disassembly and redistribution of FUS back to the nucleus during a recovery phase (i.e., after. removal of stress). GFP-FUS expressing cells will be exposed to hyperosmotic levels of sorbitol and co-localization of FUS with stress granule marker proteins (e.g., G3BP; FIG. 7A) will be quantified using ImageJ as described. We anticipated that variants altering LLPS will alter FUS granule assembly and disassembly as well as egress/re-entry. Of note, we have not observed an effect of the GFP tag on FUS assembly in these assays, and do not expect the tag or exogenous expression of FUS to confound these studies.

### Alternative Strategies:

If GFP-FUS wild-type is found to have different nuclear egress/re-entry than available in the laboratory and fixation (as in FIG. 8).

We anticipated that the FUS LC and RGG repeats mediate disordered intra-and inter-molecular contacts in both monomeric and droplet states that involve many residue types, not only the ones identified previously. We expected mutations that do not readily phase separate will still undergo egress but not go into cytoplasmic granules and will not assemble at nuclear DNA damage sites-if instead the behavior in cells is different from in vitro, we will now know which factors control assembly distinctly in cells. We also predict that S to G variant which shows more in vitro LLPS will have enhanced assembly and enhanced splicing activity but will not aggregate in the cytoplasm as seen for some disease mutations.

In addition to the expected cell-based read-outs described above, other observed phenotypes associated with the expression of LLPS-disrupting FUS variants were identified and subsequently quantified (i.e., changes in nucleocytoplasmic FUS distribution, FUS solubility, expression level, etc.). The rationale for these studies is that a detailed description of structure and contacts formed by FUS will provide information both on the function of these repeats and much needed nuances to the “stickers and spacers” model showing the role of other residue types in LLPS and mechanism of unique tyrosine contacts. These methods will serve as templates for future studies examining the large number of phase-separating IDRs that have emerged.

### Determining the Effects of Disease-Linked Mutations on FUS LLPS in Vitro and in Cells:

The biophysical properties of FUS become altered in cells as a function of disease. Many single amino acid changes found throughout both FUS LC and FUS RGGs associated with ALS including familial ALS70. The objective of this aim is to determine how these mutations modulate LLPS, aggregation, and interaction. We will test the working hypothesis that mutations outside the NLS enhance aggregation and alter nuclear and cytoplasmic FUS assembly. The rationale for this was determining how missense mutations alter the aggregation pathway will provide a target for interrupting aggregation and dysfunction.

### Justification and Feasibility

Although FUS contains many disease-associated mutations, research efforts have primarily focused on mutations in the C-terminal region that disrupt the nuclear localization signal (i.e., NLS mutations). NLS mutations in FUS prevent proper cellular localization and cause accumulation of FUS in the cytoplasm. Conversely, ALS mutations in the LC and RGG domains do not induce an obvious change in FUS cellular localization, but rather these variants remain predominately nuclear. While some LC and RGG ALS-linked variants exhibit increased aggregation propensity in vitro and when overexpressed in cells and model organisms, their mechanism of pathogenicity remains underexplored compared to NLS mutations of FUS-and may not depend on aggregation but rather on disrupted liquid-like self-interaction. Recent work examining the biophysical details of some FUS mutations are emerging using single molecule biophysics and solid-state NMR, although it remains incompletely known if and how disease-associated mutations in the LC and RGG domains alter FUS biophysical in-cell properties. Indeed, these studies are novel and will provide a new window through which we can examine the process of FUS-mediated disease.

### Research Design and Preliminary Studies:

Mechanism Underlying Mutations that Cause Disease:

Here, we analyzed the impact of mutations on FUS LC and RGG using a combination of NMR spectroscopy and microscopy, including in-cell fluorescence microscopy. We will test the hypothesis that the mutations do not alter the global disordered structure of monomers but enhance self-interaction and aggregation. These studies will provide molecular insight into the mechanism of how this self-interaction is enhanced-importantly, each mutation may cause enhanced interaction through a different mechanism. To this end, we examined FUS LC and RGG mutations (see FIG. 1), focusing on the seven LC and RGG mutations with the strongest evidence for a causal connection to ALS-being identified in cases of familial ALS (S96del, G156E, G174-175del, G191S, R234C, S462F).

### Effect of FUS Mutations on Phase Separation, Aggregation, and In-Cell Stress Response:

NLS mutations in FUS were the first to be identified in humans with ALS and were shown to induce an obvious cytoplasmic mis-localization of FUS. An analogous overt cellular phenotype has not been uncovered for variants with LC and RGG mutations, and despite their repeated occurrence in humans with familial ALS, these variants remain understudied. Therefore, our work provided the first systematic examination of the impact of these mutations on LLPS and in-cell FUS self-assembly behavior.

Based on previous observations that the G156E mutation causes FUS aggregation in the nucleus, we hypothesize that at least a subset of these non-NLS mutations will cause nuclear FUS aggregation or nuclear dysfunction. To test this hypothesis, we subjected the aforementioned LC and RGG FUS variants to the same assays previously described. Specifically, we will assess the nuclear distribution, self-assembly pattern and solubility of these variants when transfected into HeLa cells.

We also examined whether these variants self-assemble at sites of laser-induced DNA damage (FIG. 7B) and if they translocated from the nucleus to the cytoplasm into sorbitol-induced stress granules (FIG. 7A) and whether they return to their pre-damage/stress localization as observed for wild-type (FIG. 8). Given that these variants may exhibit aberrant LLPS, we may find that these variants are less likely to dissociate from each other, which could further exacerbate their putative aggregation propensities. Taken together, these studies could uncover pathogenic features of non-NLS FUS variants under both homeostatic and disease-relevant stress conditions.

### Probing Biophysical Mechanism in Vitro:

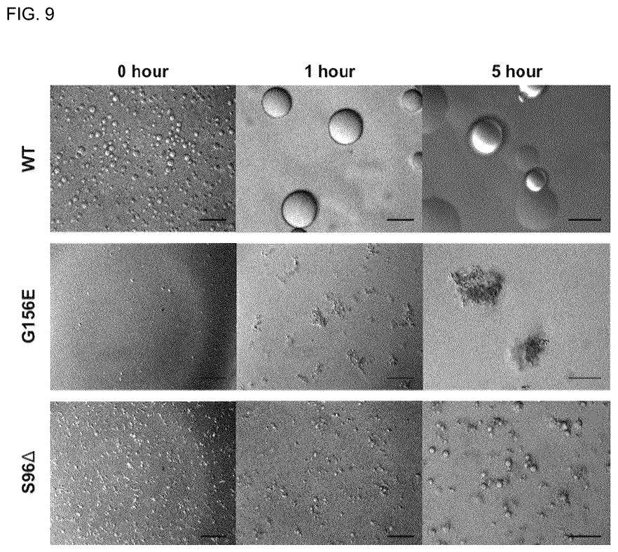

For mutations that show in-cell disruption (and for G156E which has already been shown by others to do so), we evaluated the impact of the mutations on aggregation by observing the conversion of liquid droplets to irregularly shaped aggregates by microscopy. Although this has been performed for some mutations, most work has focused on one or two non-NLS variants. In our preliminary data, we demonstrated that G156E and S96del mutations of FUS LC cause aggregation at conditions where FUS LC wild-type phase separated (FIG. 9). Presence of fibrillar structure will be evaluated by transmission electron microscopy following our published approaches. To quantify aggregation extent, we used established 1) pFTAA dye-binding assays that distinguish FUS aggregation from LLPS and 2) variable speed centrifugation to measure protein amount in pellet and supernatant. For variants that did not induce aggregation, phase separation using our assay measuring the amount of protein remaining in the supernatant (which reports on the saturation concentration) after inducing LLPS will be performed to test the hypothesis that mutations alter LLPS. Variants that show induction of aggregation in their own domain (LC or RGG) will then be evaluated for impact of the mutation on full-length FUS. Variants that do not alter biophysical behavior will not be pursued.

### Mechanism of Biophysical Change:

For each of the mutations that induce in vitro aggregation and show in-cell phenotype, we compared other variants at that position (for example for G156E, we compared G156Q/S/A/P, as recently described) to provide insight on why the observed deviation from wild-type behavior. To test the hypothesis that mutations alter local conformation, we probed similar mutations or deletions at different sites such as how we find that G51E and G101E, which are also in GYGQ-motifs like G156E, also show aggregation (in process). Finally, using variants with proline mutations at select positions, we assessed if β-sheet formation occurring at that site is essential for aggregation, and if it depends on the disease mutation.

### Probing Structural Features of Aggregation Pathway:

For the variants that enhance aggregation, we examined FUS droplets and aggregates in average structural detail following our established collaborative approach of broadband coherent anti-Stokes Raman spectroscopy (CARS) which enables probing of secondary structure with ˜300 nm spatial microscopy resolution. For G156E, our CARS data indicated FUS induces β-sheet structure formation within the aggregate. To test the hypothesis that aggregation results in contact formation in the local vicinity of the mutation, we used the pulse labeling quenched hydrogen/deuterium exchange NMR approach refined by Dobson and Robinson.

Detection of total protein mass by electrospray ionization MS provideed a measure of the distribution of exchange-protected residues as a function of time in the heterogeneous aggregates while position specific HX will be measured by HSQC NMR.

To complement the “static” view, we also used solution NMR to interrogate the aggregates. We have just observed new resonances (at distinct chemical shift positions) arising from G156E aggregates.

We further optimizde these approaches using ²H/¹⁵N/¹³C labeled protein and ¹H-¹⁵N TROSY-based 3D Cα/Cβ assignment and N spin relaxation experiments to evaluate the changes in structure and motions, respectively, for these resonances of the aggregated form. We monitored for the presence and growth of local structured regions within the aggregates of other FUS mutants using CP magic angle spinning solid state

NMR of condensed phases of ¹⁵N/¹³C FUS LC or RGG fragments and track mobile regions using INEPT experiments over time. We note these studies establish if and where conversion to β-sheet structure is present for other mutations than G156E.

To complement the NMR spectroscopy data on monomer and aggregated states, we conducted single-chain and two-chain atomistic simulations of disease mutants to identify the changes in molecular interactions and minor (if any but not detectable in experiment) structural changes upon intermolecular interactions. CG and all-atom slab simulations were used to probe intermolecular contacts and dynamics in the liquid-like assemblies of confirmed aggregation-prone mutants. These data provided critical information on differences in the contacts formed in solid-like vs. liquid-like assemblies.

Together, these results are significant because they provide a comprehensive view of which mutations alter FUS aggregation and an atomic level view of FUS mutant aggregation, both essential pieces of information to develop new strategies to inhibit the formation and toxicity of these structures.

### Alternative Strategies:

HDX NMR (and MS) relies on solubilizing aggregates in aprotic solvents to quench HDX during readout. If we were not successful in solubilizing FUS under HDX-quenching conditions, we would adapt the approach to covalently modify only exposed tyrosines spread across the entire protein with N-acetylimidazole. Modified sites wereread out 1) by chymotrypsin digest followed by mass spectrometry or 2) by 3D NMR where the modification-induced spectral changes will be correlated to resolved backbone resonances using ¹H-TOCSY-HSQC and/or H(CC)(CO)NH and (H)CC(CO)NH spectra.

A cellular picture of the impact of FUS LC and RGG mutations in FUS provides important insight into the biophysical basis for FUS dysfunction in neurodegenerative disease. Evidence how disease mutations disrupt nuclear or cytoplasmic assembly will provide essential support for the hypothesis that FUS disruptions directly lead to disease in both familial and sporadic forms of ALS and FTD. Detailed structural characterization of the monomeric forms of these variants using high-resolution NMR techniques will reveal the underlying reason for the observed change in aggregation properties. With this information, molecular strategies to block key aggregation nucleating regions can be pursued.

FUS domains cooperate in contacts with binding partners: seeing FUS in physiological context

Introduction. The RGG and LC domains of FUS play essential roles in physiological RNA interactions involved in RNA processing86. The LC and RGG domains may also mediate contacts with the disordered C-terminal domain of RNA polymerase II (CTD), stabilizing complexes of FUS in RNA transcription initiation. The objective was to bring FUS structure into physiological context—determine the dynamic structural ensemble of CTD complexes with assembled forms of FUS and probe dynamic contacts of FUS with RNA and its regulation by arginine post translational modifications. The rationale for these studies was that a detailed description of how FUS binds representative physiological targets (RNA and RNA pol II CTD) in phase-separated assemblies brings 1) details needed to understand disordered domain interactions in transcriptional activation and 2) insight into how FUS domains coordinate RNA processing functions.

FUS RGGs: RNA Binding, Arginine Modifications, and Phase Separation with RNA:

RGG repeats mediate protein-protein contacts in FUS12 but they also can interact with RNA with little RNA sequence specificity. The role of RNA interactions with FUS has begun to be probed by biochemical means, but the regions and residues that mediate contact are not clear. Do only the arginine residues mediate RNA contacts? Do the negatively charged positions in RGG contribute to binding or serve to break otherwise continuous binding regions? Furthermore, it has been reported that FUS arginine Methylation and citrullination lead to FUS dysfunction, possibly by boosting protein-protein interaction leading to aggregation. Yet, arginine methylation is known to alter FMRP protein-RNA binding, though this has not been probed for FUS. Finally, we have shown that addition of RNA stimulates FUS phase separation, but we still do not know which contacts are formed with RNA in condensed phases.

### Research Design and Preliminary Studies:

FUS RGG-RNA interactions. We probed how RGG domains bind RNA and evaluate the hypothesis that RGG methylation and citrullination modifications modulate FUS contacts.

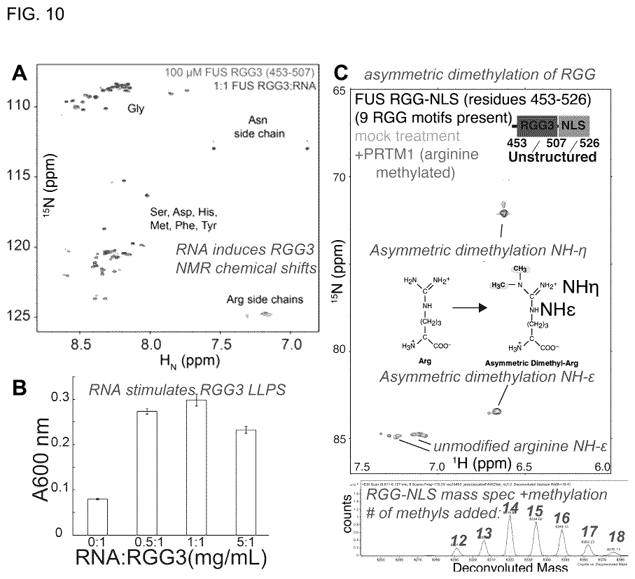

Using a total yeast RNA extract, we showed that the RGGs of FUS bind RNA, and induce chemical NMR shift changes and LLPS (FIGS. 10A,B). We also purified NMR-quantities of FUS RGGs extensively asymmetrically dimethylated at RGG positions using in vitro methylation with recombinant PRMT1 following published approaches (FIG. 10C). We next used NMR titration to probe which sites on FUS RGGs mediate contacts with total yeast extract as well as polyA, polyU, polyC, and polyG mRNA. We also tested two Bdnf RNA splice junction sequences (24 nucleotides, synthesized), which have FUS-dependent splicing in cells and which Shorter's preliminary data show disrupt FUS phase separation (in process). We used turbidity assays to probe how these distinct “generic” and specific RNA sequences compare in inducing FUS RGG phase separation (in analogy to our recent work on SARS-CoV-2 nucleocapsid protein phase separation). In addition, we evaluated how methylation of FUS RGGs alters this binding and RNA-induced phase separation as models for interactions.

RNA-RGG interactions in condensed phases. Prior work and our preliminary data demonstrated that FUS GGs bind RNAs. But how do RGGs mediate contacts with RNAs within condensed phases? Do new modes or features of interactions emerge due to the unusual environment of the phase? To take the above approaches an additional step towards reconstituting the type of assemblies observed in phase separated cellular membrane-less organelles, we used NMR to examine macroscopic condensed phases as we have demonstrated for other segments of FUS, here adding RNA. Our preliminary data demonstrated we can make macroscopic samples containing both protein and RNA.

We will now make samples of ¹⁵N/¹³C labeled protein with unlabeled RNA, beginning with polyA RNA which is known to interact with RGG domains of FUS. This labeling allowed us to examine where in the protein contacts are made with RNA via filtered/edited NMR NOE experiments as described.

In cell Splicing FUS Function:

We evaluated the impact of disruption of these RGG domain contacts on FUS RNA processing-by testing R to K substitutions as well as other contacts elucidated above. Importantly, FUS ALS-mutations in the NLS that result in cytoplasmic mis-localization disrupt FUS function in these assays, so these will serve as controls. First, we performed the established human β-globulin pDUP minigene splicing assay in HEK293 cells that measures FUS function in RNA splicing. Second, we used RT-PCR to evaluate FUS protein level autoregulation by FUS repression of exon 7 inclusion (and hence non-sense mediated decay) in endogenous FUS pre-mRNA in HEK293 cells. In these assays, disease associated variants in FUS was tested, providing further characterization of the impact of LC and RGG mutations in FUS physiological role. We expect to find that, in addition to R to K variants, mutations to RGG at G and D residues will alter RNA splicing.

Structural details of CTD interaction within FUS liquid phases. We showed that human RNA polymerase II CTD interacts not only with fibrillar forms of FUS LC assembled into amyloid/fibrillar hydrogels, but also partitions into liquid FUS LC droplets.

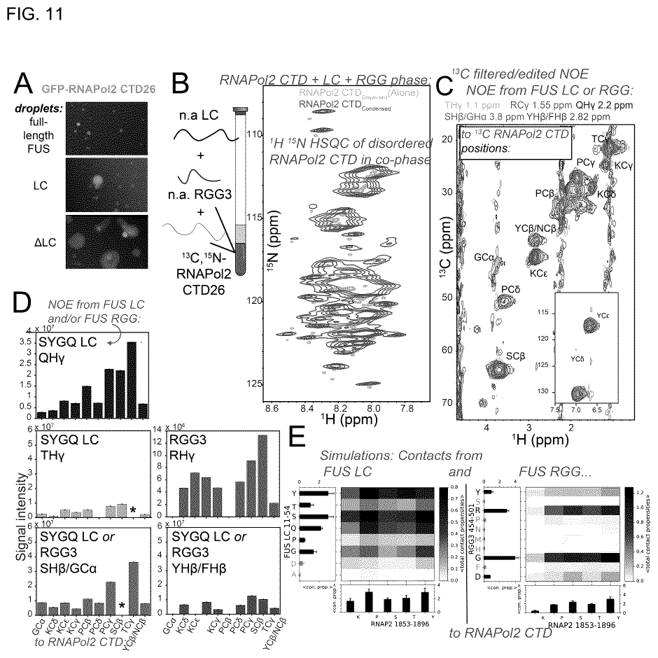

Here, our preliminary data demonstrate that CTD also partitions into liquid droplets of full-length FUS and FUS ΔLC (without the LC), suggesting that CTD makes contacts with multiple domains of FUS (FIG. 11A). To establish the nature of these contacts, we performed extensive NMR experiments on macroscopic samples of a condensed phase composed of natural isotopic abundance FUS LC and FUS RGG3 mixed with ¹⁵N/¹³C RNA Pol II CTD26 suitable for solution NMR characterization of contacts formed by RNA Pol II CTD (FIG. 11B).

These samples work towards modeling the interactions present in native transcriptional puncta where all domains of FUS are present (in some transcriptionally activating FUS cancer fusions nearly all of FUS is fused and FUS RGG domain(s) is required for oncogenic transformation in some cell types). Using this approach, we conducted 3D filtered/edited NOE experiments (FIG. 11C) (as we and others have described previously and in Aim 1) for FUS LC/RGG contacts with Pol II CTD. These data showed that many residue types in FUS LC (including Q and T, which can be unambiguously located in FUS LC) and FUS RGG3 (including R, unambiguously in RGG) show NOEs, suggestive of close contact with CTD (FIG. 11D).

Unfortunately, LC and RGG3 were both enriched in the other residue types including glycine and tyrosine. Therefore, the NOEs to CTD we observed for these residues may arise from either LC or RGG3 and we cannot compare how the sequence context (e.g. RGGYD vs SYGQ) determines contacts. To overcome this spectral/residue-type overlap, we created samples with alternate labeling patterns, including mixing ¹⁵N CTD with ¹³C FUS LC+²H FUS RGG3 to directly identify more CTD: LC contacts hotspots (see Aim 1) using ¹³C HSQC-NOESY-¹⁵N HSQC and 3D ¹³C-filtered/edited experiments, in analogy to our previous work (²H removes ¹H-¹ H NOE contributions to simplify spectra). Similarly, we used CTD samples with ²H FUS LC+¹³C RGG3 to focus on CTD: RGG contacts.

To help interpret the heterogeneity of residue contacts we observe via NMR, we performed molecular simulations of CTD1853-1896, a 44-residue fragment representing the degeneracy of CTD heptads. Interestingly, these contacts in the simulations suggested that lysines in the CTD, which we previously demonstrated are important for interaction with fibrillar polymers of the FUS-family protein TAF1580, form contacts with FUS LC and RGG3 (FIG. 11E). Using fluorescent partitioning assays with Alexa-tagged CTD variants, our data suggested that replacing lysine with CTD-consensus serine decreases partitioning into FUS LC phases. To test the role of other residues in CTD interactions, we will perform this assay with variants of FUS LC (reduced and enhanced Q content), FUS ΔLC (which removes the LC but undergoes LLPS (FIG. 11A), and RNA Pol II CTD (altering proline, serine, and threonine content). Using simulations, we extended our approach to generate atomistic condensed phase slabs with representative segments of FUS LC, FUS RGG, and RNA Pol II CTD to help interpret the experimental NOE data and provide additional contacts to test in vitro.

To test the in-cell relevance of these contacts, we tested if FUS variants that alter CTD interaction in vitro alter FUS dependent recruitment of Halo-tagged Pol II CTD to nuclear puncta in U2OS cells with genomic LacO arrays and expressing FUS-LacI fusions, which has been performed with wild-type FUS and Pol II CTD sequences previously. To probe the link to function, we tested impact of FUS variants on established FUS transcriptional activation luciferase assays (where FUS is fused to a Gal4 DNA binding domain reporter).

Seeing FUS in physiological context enabled a newly detailed understanding of FUS in the formation of functional higher order complexes. These studies provided structural information on fundamental interactions of FUS in transcription and RNA processing. We anticipated that FUS contacts other than Y and R (e.g., Q, G, and D) will make essential contributions to both RNA interaction and transcription.

### Example 2

A patient with ALS is treated with a composition disclosed herein to express an RNA-binding protein with increased proline content as compared to wild type. Progression of the disease is halted.

### Example 3

A patient with ALS is treated with a composition disclosed herein to express an RNA-binding protein with increased proline content as compared to wild type. Symptoms of the disease are decreased.

### Example 4

A patient with FTD is treated with a composition disclosed herein to express an RNA-binding protein with increased proline content as compared to wild type. Progression of the disease is halted.

### Example 5

A patient with FTD is treated with a composition disclosed herein to express an RNA-binding protein with increased proline content as compared to wild type. Symptoms of the disease are decreased.

What is claimed is:

1. A method of preventing aggregation of an RNA-binding protein comprising:
a. altering the amino acid structure of the RNA-binding protein as compared to wild type; and
b. expressing the protein in a subject.

2. The method of claim 1, wherein said method comprises identifying aggregation-prone regions and altering the proline content in said regions.

3. The method of claim 2, wherein said proline content is increased to 5%, 10%, 20%, 30%, 40%, 50%, 60%, 70%, or 80% of the sequence or sequence region as compared to wild type.

4. A method of limiting ALS or frontotemporal dementia disease progression by preventing aggregation of RNA-binding proteins comprising:
a. altering the amino acid structure of the RNA-binding protein as compared to wild type; and
b. expressing the protein in a subject.

5. The method of claim 4, wherein said method comprises identifying aggregation-prone regions and altering the proline content in said regions.

6. The method of claim 5, wherein said proline content is increased by 5%, 10%, 20%, 30%, 40%, 50%, 60%, 70%, or 80% as compared to wild type.

7. A method of limiting cancer progression by preventing aggregation of RNA-binding proteins comprising:
a. altering the amino acid structure of the RNA-binding protein as compared to wild type; and
b. expressing the protein in a subject.

8. The method of claim 7, wherein said method comprises identifying aggregation-prone regions and altering the proline content in said regions.

9. The method of claim 8, wherein said proline content is increased to 5%, 10%, 20%, 30%, 40%, 50%, 60%, 70%, or 80% of the sequence or sequence region as compared to wild type.

10. The method of claim 1, wherein said expressing the protein comprises gene therapy, mRNA delivery, or CRISPR techniques.
